(19) United States
(12) Patent Application Publication
(10) Pub. No.: US 2009/0131351 A1
(43) Pub. Date: May 21, 2009

(54) Methods, compositions, and kits for modulating tumor cell proliferation

(75) Inventors: Colin Green, London (GB); David Jones, London (GB)

Correspondence Address: MORGAN, LEWIS & BOCKIUS LLP (SF), One Market, Spear Street Tower, Suite 2800, San Francisco, CA, 94105, US

(73) Assignee: Antisoma Research Limited, London (GB)

(21) Appl. No.: 11/985,827

(22) Filed: Nov. 16, 2007

(51) Int. Cl. A61K 31/711; C12N 5/02

(52) U.S. Cl. 514/44; 435/375

(57) ABSTRACT

The present invention relates to compositions, methods, and kits for modulating tumor proliferation using G-rich oligonucleotides and one or more chemotherapeutic agents.

### CROSS-REFERENCES TO RELATED APPLICATIONS

The present application is a continuation-in-part of U.S. patent application Ser. No. 09/958,251, filed Feb. 27, 2002, which application is a National Stage of International Application No. PCT/US00/09311, filed Apr. 7, 2000 and published as WO 00/61597, which application claims the benefit of U.S. Provisional Patent Application No. 60/128,316, filed Apr. 8, 1999, and the benefit of U.S. Provisional Patent Application No. 60/149,823, filed Aug. 19, 1999, the contents of each of which are incorporated herein by reference in their entirety for all purposes. The present application is related to U.S. patent application Ser. No. 10/978,032, filed on Oct. 29, 2004, and U.S. patent application Ser. No. ______, filed Oct. 31, 2007, the contents of which is incorporated herein by reference in its entirety for all purposes.

### FIELD OF THE INVENTION

The present invention relates generally to methods and compositions for modulating tumor cell proliferation.

### BACKGROUND OF THE INVENTION

Oligonucleotides have the potential to recognize unique sequences of DNA or RNA with a remarkable degree of specificity. For this reason they have been considered as promising candidates to realize gene specific therapies for the treatment of malignant, viral and inflammatory diseases. Two major strategies of oligonucleotide-mediated therapeutic intervention have been developed, namely, the antisense and antigene approaches.

The antisense strategy aims to down-regulate expression of a specific gene by hybridization of the oligonucleotide to the specific mRNA, resulting in inhibition of translation. Gewirtz et al. (1998) Blood 92, 712-736; Crooke (1998) Antisense Nucleic Acid Drug Dev. 8, 115-122; Branch (1998) Trends Biochem. Sci. 23, 45-50; Agrawal et al. (1998) Antisense Nucleic Acid Drug Dev. 8, 135-139.

The antigene strategy proposes to inhibit transcription of a target gene by means of triple helix formation between the oligonucleotide and specific sequences in the double-stranded genomic DNA. Helene et al. (1997) Ciba Found. Symp. 209, 94-102.

Whereas both the antisense and antigene strategies have met with some success, it has become clear in recent years that the interactions of oligonucleotides with the components of a living organism go far beyond sequence-specific hybridization with the target nucleic acid. Recent studies and re-examination of early antisense data have suggested that some of the observed biological effects of antisense oligonucleotides cannot be due entirely to Watson-Crick hybridization with the target mRNA. In some cases, the expected biological effect (e.g. inhibition of cell growth or apoptosis) was achieved, but this was not accompanied by a down regulation of the target protein and unlikely to function through the antisense mechanism. White et al. (1996) Biochem. Biophys. Res. Commun. 227, 118-124; Dryden et al. (1998) J. Endocrinol. 157, 169-175.

In many cases, it was demonstrated that other non-sequence-specific oligonucleotides could exert biological effects that equalled or exceeded the antisense sequence. Barton et al. (1995) Br. J. Cancer 71, 429-437; Burgess et al. (1995) Proc. Natl. Acad. Sci. U.S.A. 92, 4051-4055; Benimetskaya et al. (1997) Nucleic Acids Res. 25, 2648-2656.

Aptamers are amongst such non-sequence-specific oligonucleotides. These oligonucleotides can bind to a specific molecular partner through intramolecular or intermolecular interactions that fold the molecule into a complex tertiary structure, forming intramolecular or intermolecular structures that allow the aptamer(s) to bind stably to their target molecules. See Osborne et al., 1997, Curr. Opin. Chem. Biol. 1:5-9; Patel, 1997, Curr. Opin. Chem. Biol. 1:32-46.

Such structures are thought to play an important role in vivo and putative quartet forming sequences have been identified in telomeric DNA (Sundquist et al. (1989) Nature 342, 825-829), immunoglobulin switch region sequences (Sen et al. (1988) Nature 334, 364-366), HIV1 RNA (Sundquist et al. (1993) Proc. Natl. Acad. Sci U.S.A. 90, 3393-3397), the fragile X repeat sequences (Fry et al (1994) Proc. Natl. Acad. Sci. U.S.A. 91, 4950-4954) and the retinoblastoma gene (Murchie et al. (1992) Nucleic Acids Res. 20, 49-53)

Since nucleic acid molecules are typically more readily introduced into target cells than therapeutic protein molecules are, aptamers offer a method by which proliferative activity can be suppressed. Studies have shown that the administration of oligonucleotides can be administered in a clinically relevant way and have relatively few toxic side effects. See Gewirtz et al. (1998) Blood 92, 712-736; Agrawal et al. (1998) Antisense Nucleic Acid Drug Dev. 8, 135-139.

Applicants have previously described G-rich oligonucleotides (GROs) that have potent growth inhibitory effects that are unrelated to any expected antisense or antigene activity. The antiproliferative effects of these oligonucleotides have be identified by the applicants as being related to their ability to bind to a specific cellular protein. Because the GRO binding protein is also recognized by antinucleolin antibodies, Applicants have concluded that this protein is either nucleolin itself, or a protein of a similar size that shares immunogenic similarities with nucleolin.

Nucleolin is an abundant multifunctional 110 kDa phosphoprotein thought to be located predominantly in the nucleolus of proliferating cells (for reviews, see Tuteja et al. (1998) Crit. Rev. Biochem. Mol. Biol. 33, 407-436; Ginisty et al. (1999) J. Cell Sci. 112, 761-772). Nucleolin has been implicated in many aspects of ribosome biogenesis including the control of rDNA transcription, pre-ribosome packaging and organization of nucleolar chromatin. Tuteja et al. (1998) Crit. Rev. Biochem. Mol. Biol. 33, 407-436; Ginisty et al. (1999) J. CellSci. 112, 761-772; Ginisty et al. (1998) EMBO J. 17, 1476-1486.

Another role for nucleolin is as a shuttle protein that transports viral and cellular proteins between the cytoplasm and nucleus/nucleolus of the cell. Kibbey et al. (1995) J. Neurosci. Res. 42, 314-322; Lee et al. (1998) J. Biol. Chem. 273, 7650-7656; Waggoner et al. (1998) J. Virol. 72, 6699-6709.

Nucleolin is also implicated, directly or indirectly, in other roles including nuclear matrix structure (Gotzmann et al. (1997) Electrophoresis 18, 26452653), cytokinesis and nuclear division (Léger-Silvestre et al. (1997) Chromosoma 105, 542-52), and as an RNA and DNA helicase (Tuteja et al. (1995) Gene 160, 143-148).

The multifunctional nature of nucleolin is reflected in its multidomain structure consisting of a histone-like N-terminus, a central domain containing RNA recognition motifs, and a glycine/arginine rich C-terminus. Lapeyre et al. (1987) Proc. Natl. Acad. Sci. U.S.A. 84, 1472-1476. Levels of nucleolin are known to relate to the rate of cellular proliferation (Derenzini et al. (1995) Lab. Invest. 73, 497-502; Roussel et al. (1994) Exp. Cell Res. 214, 465-472.), being elevated in rapidly proliferating cells, such as malignant cells, and lower in more slowly dividing cells.

Various chemotherapeutic agents are currently in use for the inhibition of tumor growth associated with various cancers. For instance, cytarabine (“cytosine arabinoside” or also known as “Ara-C”) is commonly used in cancer treatment, e.g. head and neck cancer, leukaemia and non-Hodgkin lymphoma. Cytarabine is an anti-metabolic agent with the chemical name of 1β-arabinofuranosylcytosine:

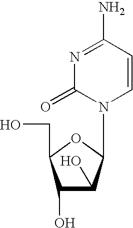

Its mode of action is due to its rapid conversion into cytosine arabinoside triphosphosphate, which damages DNA when the cell cycle holds in the S phase (synthesis of DNA). Rapidly dividing cells, which require DNA replication for mitosis, are therefore most affected. Cytarabine also inhibits both DNA and RNA polymerases and nucleotide reductase enzyme needed for DNA synthesis.

Cytarabine is available commercially from Sigma-Aldrich Company Limited (Dorset, UK; catalogue number C-6645) and is also available as a generic product under the names Cytosar-U and Tarabine PFS.

Cytarabine is typical of most chemotherapeutic agents in that it is not very selective in the targets it acts upon, thereby causing serious side-effects. Examples of side-effects of cytarabine include bone marrow suppression, cerebellar toxicity when given in high doses, leukopenia, thrombocytopenia, anaemia, GI disturbances, stomatitis, conjunctivitis, pneumonitis, fever, and dermatitis.

What is needed are methods and agents that modulate tumor cell proliferation with improved efficacy and reduced toxicity. The present invention provides agents that can be administered alone or in combination with conventional chemotherapeutics to enhance their function and/or permit reduced dosages and minimized toxicity ensuing from the use of conventional chemotherapeutics.

### SUMMARY OF THE INVENTION

The present invention provides methods, compositions, and kits for modulating tumor cell proliferation. Aptamers of the invention can be administered in combination with other chemotherapeutics, such as cytarabine, to achieve a synergistic effect in the inhibition of tumor cell proliferation.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended Figures. These Figures form a part of the specification. It is to be noted, however, that the appended Figures illustrate preferred embodiments of the invention and therefore are not to be considered limiting in their scope.

FIG. 1 shows the absorbance in a Sulphorhodamine B assay after incubation of AML cells with AS1411 and cytarabine according to an exemplary embodiment of the invention.

FIG. 2 shows the absorbance in a Sulphorhodamine B assay after incubation of different cell lines with AS1411 according to an exemplary embodiment of the invention.

FIG. 3 illustrates a preferred ambulatory device for use in the administration regimen (the Baxter FOLFusor LV10) according to an exemplary embodiment of the invention.

FIG. 4 illustrates the relative tumor volume at 4 and 7 days post-treatment (relative to tumor volume at Day 0 of treatment) in xenograft mouse model according to an exemplary embodiment of the invention. Data is also presented in Table 3 of the following disclosure.

FIG. 5 illustrates the distribution of tumor volume change in xenograft mouse model at 7 days post-treatment according to an exemplary embodiment of the invention. Data is also presented in Table 2 of the following disclosure.

### DETAILED DESCRIPTION OF THE INVENTION

### Definitions

It is to be understood that this invention is not limited to the particular methodology, protocols, cell lines, animal species or genera, constructs, and reagents described, as such may vary. It is also to be understood that the terminology used herein is for the purpose of describing particular embodiments only, and is not intended to limit the scope of the present invention which will be limited only by the appended claims. Where not specifically set forth, terminology used herein should be construed in accordance with their meaning in the art.

As used herein the singular forms “a”, “and”, and “the” include plural referents unless the context clearly dictates otherwise. For example, “a compound” refers to one or more of such compounds, while “the enzyme” includes a particular enzyme as well as other family members and equivalents thereof as known to those skilled in the art.

“Hyperproliferative disorder” refers to excess cell proliferation, relative to that occurring with the same type of cell in the general population and/or the same type of cell obtained from a patient at an earlier time. The term denotes malignant as well as non-malignant cell populations. Such disorders have an excess cell proliferation of one or more subsets of cells, which often appear to differ from the surrounding tissue both morphologically and genotypically. The excess cell proliferation can be determined by reference to the general population and/or by reference to a particular patient, e.g. at an earlier point in the individual's life. Hyperproliferative cell disorders can occur in different types of animals and in humans, and produce different physical manifestations depending upon the affected cells.

Hyperproliferative cell disorders include cancers. Cancers are of particular interest, including leukemias, lymphomas (Hodgkins and non-Hodgkins), and other myeloproliferative disorders; carcinomas of solid tissue, sarcomas, melanomas, adenomas, hypoxic tumors, squamous cell carcinomas of the mouth, throat, larynx, and lung, genitourinary cancers such as cervical and bladder cancer, hematopoietic cancers, head and neck cancers, and nervous system cancers, benign lesions such as papillomas, and the like.

As used herein, the term “neoplastic” includes the new, abnormal growth of tissues and/or cells, such as a cancer or tumor, including, for example, breast cancer, leukemia or prostate cancer. The term “neoplastic” also includes malignant cells which can invade and destroy adjacent structures and/or metastasize.

As used herein, the term “dysplastic” includes any abnormal growth of cells, tissues, or structures including conditions such as psoriasis.

As used herein, the term “aptamer analog” or “analog of an aptamer” refers to a variant oligonucleotide, including RNA and DNA, wherein one or more residues of the reference aptamer has been substituted by other residue(s); wherein one or more residues, natural or synthetic, have been deleted from the reference aptamer sequence; and further includes aptamers having additional residues to the reference sequence and said variant oligonucleotide has a tertiary structure that can bind specifically to the same binding partner of the reference aptamer. The residues referred to above may be natural or modified/synthetically formed. Armed with the guidance of the present disclosure, those of ordinary skill in the art will be able to identify analogs using the systematic evolution of ligands by exponential enrichment (SELEX) process, which allows for the isolation of oligonucleotide sequences with the capacity to recognize virtually any class of target molecules with high affinity and specificity, and other technologies currently known in the art for identifying molecules having a certain binding specificity.

As used herein, the term “metastatic” or “metastatic disease” refers to diseases which have spread to regional lymph nodes or to distant sites and includes, without limitation, cancers and malignant tumors.

An individual “afflicted with” a particular disease means that the individual individual has been diagnosed as having, or is suspected as having, the disease.

The “individual,” or “patient,” may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease. The term “patient” means an individual having a disorder in need of treatment.

As used herein, an “effective amount” (e.g., of an agent) is an amount (of the agent) that produces a desired and/or beneficial result. An effective amount can be administered in one or more administrations. For purposes of this invention, an effective amount is an amount sufficient to produce modulation of tumor cell proliferation. An “amount sufficient to modulate tumor cell proliferation” preferably is able to alter the rate of proliferation of tumor cells by at least 25%, preferably at least 50%, more preferably at least 75%, and even more preferably at least 90%.

Such modulation may have desirable concomitant effects, such as to palliate, ameliorate, stabilize, reverse, slow or delay progression of disease, delay or even prevent onset of disease.

As used herein, the term “agent” means a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic compounds based on various core structures, and these are also included in the term “agent”. In addition, various natural sources can provide compounds, such as plant or animal extracts, and the like. Agents include, but are not limited to, polyamine analogs. Agents can be administered alone or in various combinations.

“Modulating” cell proliferation means that the rate of proliferation is altered when compared to not administering an agent that, but is not limited to, interferes with the cell cycle, arrests cell-cycle, for example at the S-phase, inhibits DNA replication, induces cell death, etc. The mechanism of the present invention takes advantage of the presence of cell-surface nucleolin as a cancer marker, e.g. as observed by the Applicants in AML blasts. The binding of the modulating agents of the present invention brings about a cascade of events, including, but not limited, to uptake of the nucleolin-agent complex into the hyperproliferative cell and interference of nucleolin function in nucleus, cytoplasm and/or membrane. Preferably, “modulating” tumor cell proliferation means a change in the rate of tumor cell proliferation of at least 25%, preferably at least 50%, more preferably at least 75%, and even more preferably at least 90%. Generally, for purposes of this invention, “modulating” cell proliferation means that the rate of proliferation is decreased when compared to the rate of proliferation in that individual when no agent is administered. However, during the course of therapy, for example, it may be desirable to increase the rate of proliferation from a previously measured level. In individuals afflicted with tumors, the degree of modulation may be assessed by measurement of tumor cell proliferation, which will be discussed below, and generally entails detecting a proliferation marker(s) in a tumor cell population or uptake of certain substances which would provide a quantitative measure of proliferation. Any quantitative methods for measuring tumor cell proliferation currently known or unknown in the art can be used for this purpose. Further, it is possible that, if the cells are proliferating due to a genetic alteration (such as transposition, deletion, or insertion), this alteration could be detected using standard techniques in the art, such as RFLP (restriction fragment length polymorphism).

The term “inhibiting the proliferation of malignant, dysplastic, and/or hyperplastic cells” includes any partial or total growth inhibition and includes decreases in the rate of proliferation or growth of the cells.

“Anti-proliferative agents,” as used herein, refer to agents that modulate cell proliferation as defined herein.

The various active components of the therapeutic compositions disclosed herein are present in an “effective combination” when there are sufficient amounts of each of the components for the co-administration, be it simultaneous or timed in proximity to one another, to be effective in modulating tumor cell proliferation.

The term “treatment” as used herein refers to reducing or alleviating symptoms in an individual, preventing symptoms from worsening or progressing, modulation or elimination of the causative agent, or prevention of the disorder in an individual who is free therefrom. F or example, treatment of a cancer patient may be reduction of tumor size, elimination of malignant cells, prevention of metastasis, or the prevention of relapse in a patient whose tumor has regressed. The treatment of ongoing disease, to stabilize or improve the clinical symptoms of the patient, is of particular interest.

Those skilled in the art are easily able to identify patients having a malignant, dysplastic, or a hyperproliferative condition such as a cancer or psoriasis, respectively. For example, patients who have a cancer such as breast cancer, prostate cancer, cervical carcinomas, and the like.

A “therapeutically effective amount” is an amount of an oligonucleotide of the present invention, that when administered to the individual, ameliorates a symptom of the disease, disorder, or condition, such as by modulating or reducing the proliferation of dysplastic, hyperproliferative, or malignant cells.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence (e.g., when aligning a second sequence to the 69087 amino acid sequence of SEQ ID NO: 2, 100 amino acid residues, preferably at least 200, 300, 400, or 500 or more amino acid residues are aligned). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman et al. (1970, J. Mol. Biol. 48: 444-453) algorithm which has been incorporated into the GAP program in the GCG software package (available at www.gcg.com), using either a BLOSUM 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a BLOSUM 62 scoring matrix with a gap penalty of 12, a gap extend penalty of 4, and a frameshift gap penalty of 5.

The percent identity between two amino acid or nucleotide sequences can be determined using the algorithm of Meyers et al. (1989, CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990, J. Mol. Biol. 215:403-410). BLAST nucleotide searches can be performed with the NBLAST program, score=100, wordlength=12 to obtain nucleotide sequences homologous to 69087, 15821, or 15418 nucleic acid molecules of the invention. BLAST protein searches can be performed with the XBLAST program, score=50, wordlength=3 to obtain amino acid sequences homologous to 69087, 15821, or 15418 protein molecules of the invention. To obtain gapped alignments for comparison purposes, gapped BLAST can be utilized as described in Altschul et al. (1997, Nucl. Acids Res. 25:3389-13402). When using BLAST and gapped BLAST programs, the default parameters of the respective programs (e.g., XBLAST and NBLAST) can be used. See <www.ncbi.nlm.nih.gov>.

Exemplary oligonucleotides of the present invention are designated below:

| | |
|---|---|
| GRO14A | 5′-GTTGTTTGGGGTGG-3′ |
| | SEQ ID No: 1 |
| GRO15A | 5′-GTTGTTTGG GGTGGT-3′ |
| | SEQ ID No: 2 |
| GR025A | 5′-GGTTGGGGTGGGTGGGGTG GGTGGG-3′ |
| | SEQ ID No: 3 |
| GR028A | 5′-TTTGGTGGTGGTGGTTGTGG TGGTGGTG-3′ |
| | SEQ ID No: 4 |
| GR029A | 5′-TTTGGTGGTGGTGG TTGTGGTGGTGGTGG-3′ |
| | SEQ ID No: 5 |
| GR029- | 5′-TTTGGTGG TGGTGGTTTTGGTGGTGGTGG-3′ |
| 2 | SEQ ID No: 6 |
| GR029- | 5′-TTTGGTGGTGGTGGTGGTGGTGGTGGTGG-3′ |
| 3 | SEQ ID No: 7 |
| GR029- | 5′-TTTGGTGGTGGTGGTTTGGGTGGTGG TGG-3′ |
| 5 | SEQ ID No: 8 |
| GR029- | 5′-TGGTGGTGGTGGT-3′ |
| 13 | SEQ ID No: 9 |
| GRO11A | 5′-GGTGGTGGTGG-3′ |
| | SEQ ID No: 10 |
| GR014C | 5′-GGTGGTTGTGGTGG-3′ |
| | SEQ ID No: 11 |
| GR026B | 5′-GGTGGTGGTGGTTGTGGTGG TGGTGG-3′ |
| | SEQ ID No: 12 |
| GR056A | 5′-GGTGGTGGTGGTTG |
| | TGGTGGTGGTGGTTGTGGTGGTGGTGGTTGTGGTGGTGGTGG- |
| | 3′ |
| | SEQ ID No: 13 |
| GR032A | 5′-GGTGGTTGTGGTGGTTGTGGTGGTTGT GGTGG-3′ |
| | SEQ ID No: 14 |
| GR032B | 5′-TTTGGTGGTGGTGGTTGTGGT GGTGGTGGTTT-3′ |
| | SEQ ID No: 15 |
| GR029- | 5′-GGTGGTGGTGGTTGT GGTGGTGGTGGTTT-3′ |
| 6 | SEQ ID No: 16 |
| GR028B | 5′-TTTGGTGGTGGT GGTGTGGTGGTGGTGG-3′ |
| | SEQ ID No: 17 |
| GRO13A | 5′-TGGTGGTGGT-3′ |
| | SEQ ID No: 18 |

Other oligonucleotides having the same activity are also contemplated.

The present invention provides compositions, methods, and kits relating to G-rich oligonucleotides and a chemotherapeutic in effective combination to modulate tumor cell proliferation.

By G-rich oligonucleotide (GRO), it is meant that the oligonucleotides consist of 4-100 nucleotides (preferably 10-30 nucleotides) with DNA, RNA, 2′-O-methyl, phosphorothioate or other chemically similar backbones. Their sequences contain one or more GGT motifs. The residues employed can be naturally found or synthetically formed. The oligonucleotides have antiproliferative activity against cells and bind to GRO binding protein and/or nucleolin. These properties can be demonstrated using techniques well known in the art such as an MTT assay or the EMSA technique (see WO 2000/61597).

The oligonucleotides of the present invention are rich in guanosine and are capable of forming G-quartet structures. Specifically, the oligonucleotides of the present invention are primarily comprised of thymidine and guanosine with at least one contiguous guanosine repeat in the sequence of each oligonucleotide. The G-rich oligonucleotides disclosed herein are stable and can remain undegraded in serum for prolonged periods of time and have been found to retain their growth modulating effects for periods of at least seven days.

The GROs of the present invention can be administered to a patient or individual either alone or as part of a pharmaceutical composition. The GROs can be administered to patients either orally, rectally, parenterally (intravenously, intramuscularly, or subcutaneously), intracistemally, intravaginally, intraperitonally, intravesically, locally (powders, ointments, or drops), or as a buccal or nasal spray.

In some embodiments, the G-rich oligonucleotide has a 3′ end and a 5′ end, wherein one or both of the 3′ and 5′ ends have been modified to alter a property of the G-rich oligonucleotide.

For instance, the oligonucleotides can be modified at their 3′ end in order to alter a specific property of the oligonucleotide. In some exemplary embodiments, the 3′-terminus of the oligonucleotide can be modified by the addition of a propylamine group, which has been found to increase the stability of the oligonucleotide to serum nucleases. Likewise, the G-rich oligonucleotides of the present invention can have other modifying groups known in the art for minimizing immunogenicity or decreasing susceptibility to protease degradation. Other modifications that are well known in the art include 3′ and 5′ modifications, for example, the binding of cholesterol, and backbone modifications, for example, phosphorothioate substitution and/or 2′-O-methyl RNA.

Cytarabine is known to have activity against solid tumors (such as squamous cell carcinoma of the head and neck) but its activity against some solid tumor types is sometimes limited. Experiments conducted using cell lines of solid tumors indicate that the expression of cytidine deaminase (an enzyme that degrades cytarabine) is high, whereas the expression of deoxycytidine kinase (which phosphorylates cytarabine, and thereby increases its activity) is weak, which may account for the sometimes limited activity of cytarabine in certain cases.

The present invention provides compositions comprising a G rich oligonucleotide having a sequence selected from SEQ ID NO: 1 to 18 (or its aptamer analog) and cytarabine in effective combination. In some embodiments of the invention, the compositions further include a pharmaceutically acceptable excipient, diluent or carrier. In exemplary embodiments, two or more G-rich oligonucleotides having a sequence selected from SEQ ID NO: 1-18 or their aptamer analogs are used in effective combination in the compositions.

In preferred embodiments, the compositions comprise a G-rich oligonucleotide having the sequence of SEQ ID NO: 12 or its aptamer analog.

Also provided is a kit useful for modulating tumor cell proliferation comprising a G-rich oligonucleotide having a sequence selected from SEQ ID NO: 1-18 or its aptamer analog; and cytarabine in effective combination; and instructions for their use.

In some embodiments, the kit further comprises a system for administering the G-rich oligonucleotide and/or cytarabine to a patient. Systems for administering pharmaceutical compositions to patients known in the art can be employed for the purpose disclosed herein. In exemplary embodiments of the invention, the system includes an ambulatory device, such as the Baxter FOLFusor LV10.

In preferred embodiments, the G-rich oligonucleotide and cytarabine are contained separately until use. Alternatively, the G-rich oligonucleotide and cytarabine are provided as an admixture. In preferred embodiments, the G-rich oligonucleotide included in the kit has the sequence of SEQ ID NO: 12 or is its aptamer analog.

In some embodiments of the kit, the G-rich oligonucleotide has a 3′ end and a 5′ end, wherein one or both of the 3′ and 5′ ends have been modified to alter a property of the G-rich oligonucleotide.

The present invention also provides methods for inhibiting the proliferation of malignant, dysplastic, and/or hyperproliferative cells, said methods comprising administering to the subject a G-rich oligonucleotide having the sequence selected from SEQ ID NO: 1-18 or its aptamer analog and a chemotherapeutic agent in in effective combination. In preferred embodiments, the chemotherapeutic agent is cytarabine. In other embodiments, to achieve a synergistic effect in modulating tumor cell proliferation, the chemotherapeutic agent can be selected as paclitaxel, which has demonstrated effective combinatory effect with AS1411 in various tumor types, such as lung and breast cancers.

### Susceptible Tumors

Tumors of interest include carcinomas, e.g. colon, prostate, breast, melanoma, ductal, endometrial, stomach, dysplastic oral mucosa, invasive oral cancer, non-small cell lung carcinoma, renal cell carcinoma, transitional and squamous cell urinary carcinoma, etc.; neurological malignancies, e.g. neuroblastoma, gliomas, etc.; hematological malignancies, e.g. childhood acute leukemia, non-Hodgkin's lymphomas, and other myeloproliferative disorders, chronic lymphocytic leukemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Cancers of particular interest for treatment by the compositions disclosed herein include, without limitation, acute myelogenous leukaemia, acute myeloid leukaemia (AML), acute lymphoblastic leukaemia (ALL), chronic myelogenous leukemia (CML), lymphomas, non-Hodgkin's lymphoma, and solid tumors including squamous cell carcinoma (such as head and neck cancer, and/or squamous cell carcinoma of the head and neck).

### Modes of Administration and Dose

The compositions of this invention may be administered to the subject at any site, particularly a site that is “distal” to or “distant” from the primary tumor.

The compositions of the present invention can be administered to individuals orally, rectally, parenterally (intravenously, intramuscularly, or subcutaneously), intradermally, intracisternally, intravaginally, intraperitonally, intravesically, locally (powders, ointments, or drops), or as a buccal or nasal spray, via an afferent lymph vessel, or by another route that is suitable in view of the tumor being treated and the subject's condition. The aptamers of the present invention can be administered to an individual either alone, in combination with one another or other chemotherapeutic agents, and/or as part of a pharmaceutical composition.

Preferably the G-rich oligonucleotide and chemotherapeutic agent are administered between 0 and 24 hours apart with either the oligonucleotide or the chemotherapeutic being administered first. In some embodiments, the administration of the G-rich oligonucleotide precedes administration of the chemotherapeutic agent. In other embodiments, the chemotherapeutic agent treatment precedes treatment with the G-rich oligonucleotide. In still other embodiments, the G-rich oligonucleotide and the chemotherapeutic agent are administered simultaneously.

The agents of the present invention can be incorporated into a variety of formulations for therapeutic administration. More than one of the agents described herein can be delivered simultaneously, or within a short period of time, by the same or by different routes. In one embodiment of the invention, a co-formulation is used, where the two components are combined in a single suspension. Alternatively, the two may be separately formulated.

The present invention also encompasses methods for modulating the proliferation of tumor cells and cells demonstrating malignant, dysplastic, hyperproliferative, or metastatic activity in an individual, comprising systemically (generally, orally) administering to a subject having a nervous system, particularly a vertebrate, preferably a mammal, most preferably a human, successive therapeutically effective doses of the present compositions.

In accordance with the methods of the present invention, the composition described herein is administered to a mammal, preferably a human. Preferably, such administration is oral. As used herein, the term “oral administration” (or the like) with respect to the subject (preferably, human) means that the subject ingests or is directed to ingest (preferably, for the purpose of treatment of one or more of the various health problems described herein) one or more components of the present invention/compositions of the present invention. Wherein the subject is directed to ingest one or more of the components of the present invention/compositions, such direction may be that which instructs and/or informs the user that use of the composition may and/or will provide treatment for the particular health problem of concern. For example, such direction may be oral direction (e.g., through oral instruction from, for example, a physician, sales professional or organization, and/or radio or television media (i.e., advertisement) or written direction (e.g., through written direction from, for example, a physician or other medical professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” means through words, pictures, symbols, and/or other visible descriptors.

Administration of the present components of the invention/compositions may be via any systemic method, however, such administration is preferably oral. Exemplary modes of administration include oral, rectal, topical, sublingual, transdermal, intravenous infusion, pulmonary, intramuscular, intracavity, aerosol, aural (e.g., via eardrops), intranasal, inhalation, needleless injection, or subcutaneous delivery. Direct injection could also be preferred for local delivery. For continuous infusion, a PCA device may be employed. Oral or subcutaneous administration may be important for the convenience of the patient as well as the dosing schedule. Preferred rectal modes of delivery include administration as a suppository or enema wash. For transdermal administration, an ionopheresis device may be employed to enhance penetration of the active drug through the skin. Such devices and methods useful in ionophoresis current assisted transdermal administration include those described in U.S. Pat. Nos. 4,141,359; 5,499,967; and 6,

In some embodiments, partial doses or doses of different agents described herein are administered simultaneously or at different times by different routes. Such administration may use any route that results in systemic absorption, by any one of several known routes, including but not limited to inhalation, i.e. pulmonary aerosol administration; intranasal; sublingually; orally; and by injection, e.g. subcutaneously, intramuscularly, etc.

More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

In pharmaceutical dosage forms, the compounds may be administered in the form of their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant containing the therapeutic agent is placed in proximity to the site of the tumor, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the unit dosage forms of the present invention depend on the particular compound employed and the effect to be achieved, and the pharmacodynamics associated with each compound in the host.

Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Compositions of the present invention suitable for parenteral injection may comprise physiologically acceptable sterile aqueous or nonaqueous solutions, dispersions, suspensions or emulsions, and sterile powders for reconstitution into sterile injectable solutions or dispersions known in the art.

In some preferred embodiments, the compositions of the invention are administered intravenously, e.g. through attachment to a drip or infusion bag and any other similar means known in the art.

Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound (GRO) is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They may contain opacifying agents, and can also be of such composition that they release the active compound or compounds in a certain part of the intestinal tract in a delayed manner.

Examples of embedding compositions that can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the compositions can also include adjuvants, such as wetting agents, emulsifying and suspending agents, sweetening, flavoring, and perfuming agents.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage forms for topical administration of a GRO of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

In addition, the GROs of the present invention can exist in unsolvated as well as solvated forms with pharmaceutically acceptable solvents such as water, ethanol, and the like. In general, the solvated forms are considered equivalent to the unsolvated forms for the purposes of the present invention.

In addition, it is intended that the present invention cover GROs made either using standard organic synthetic techniques, including combinatorial chemistry or by biological methods, such as through metabolism.

### Dosage

Generally, the GROs and cytarabine of the present invention can be given in single and/or multiple dosages or administered continuously. Depending on the patient and condition being treated and on the administration route, the agent(s) of the invention can be administered in dosages of about 1-100 mg/kg per day, preferably about 10-60 mg/kg, more preferably about 1-40 mg/kg, and even more preferably about 20-40 mg/kg or about 5-10 mg/kg. Administration can occur over a period ranging from about 1-10 days, preferably 1-7 days, and more preferably about 4-7 days. Those of ordinary skill in the art will appreciate that the mode of administration can have a large effect on dosage. Thus for example oral dosages maybe ten times the injection dose. The dosage for the anti-proliferative agents will also vary with the precise compound, in accordance with the nature of the agent. Higher doses may be used for localized routes of delivery.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the individual to side effects. Some of the specific compounds are more potent than others. Preferred dosages for a given compound are readily determinable by those of skill in the art by a variety of means. A preferred means is to measure the physiological potency of a given compound.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### Example 1

### Effects of AS1411 (GRO SEQ ID No. 12) and Cytarabine

### Sulphorhodamine B Assay

MV4-11 cells (AML cell line available and obtained from ATCC under catalogue number CRL-9591) were seeded in wells of a 96-well plate at a number optimised for each cell line. 1 μM AS1411 was added with varying concentrations of cytarabine (0.002 to 1.5 μM) and cells were incubated for 6 days. A control was run with the 1 μM AS1411 without cytarabine. A second control series was run with the varying amounts of cytarabine but with no AS1411 present.

Cells were then washed, fixed to the 96-well plate and exposed to the dye Sulphorhodamine B (SRB; available from Sigma-Aldrich, Dorset, UK; catalogue number S-1402). The remaining cell mass after exposure to AS1411 was measured with a spectrophotometer and IC₅₀ determined.

### Results

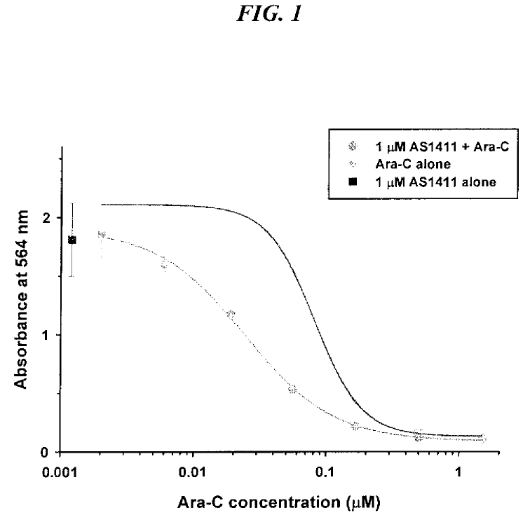

FIG. 1 shows the absorbance using the SRB dye after incubation with AS1411 and/or cytarabine. The combination index (R) is greater than 1, indicating a synergistic effect. In this particular case the IC₅₀ of cytarabine was reduced more than three-fold by combination with AS1411. Results from the study also show an induction of apoptosis, increase in caspase activity, dose-dependent increase in DNA fragmentation, and increase in PS externalization, arrest at the S-phase in tumor cells with administration of AS1411 to AML cell line.

### Example 2

### Comparative Effects of AS1411 on Different Cell Lines

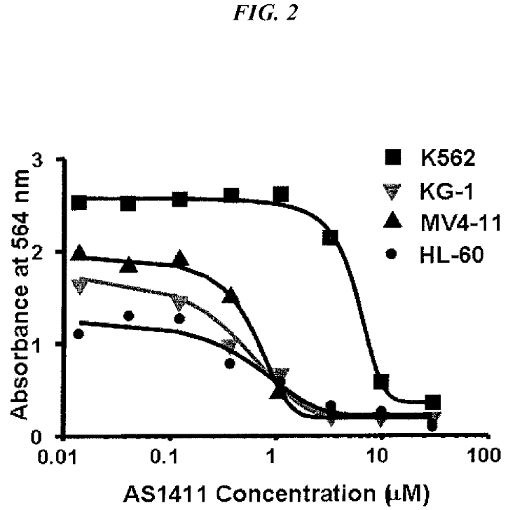

Multiple haematological cancer cell lines were exposed to varying concentrations of AS1411 over a period of 6 days according to the Sulphorhodamine B assay described in Example 1. The Acute Myelogenous Leukaemia (AML) cell lines KG-1, MV4-11 and HL-60 (all available and obtained from ATCC under catalogue numbers CCL-246, CRL-9591 and CCL-240, respectively) exhibited low IC₅₀ values demonstrating that AS1411 is effective in inhibiting growth and killing these cell lines. (See FIG. 2). Other tumor cell lines demonstrating sensitivity to AS1411 include DU-145, PC3, A549, CALU06, LS174T, HT-29, MCF-7, ZR75-1, HGC.27, KATO III, A498, PANC-1, SKOV-3, U87-MG, SK-MEL-28, which encompass tumor types ranging from prostate, lung, colon, breast, grastric, renal, pancreatic, ovarian, glioblastoma, and melanoma. Normal fibroblast and B cells were however unaffected by similar concentrations of AS1411.

### Example 3

### Use of Combination Therapy in Cancer Treatment

The combination therapy experimentally tested in example 1 can be applied to use in the treatment of human tumors.

Treatment of human tumors requires administration of the standard clinical chemotherapy dose in mg/m² (mg/m² is calculated approximately by multiplying mg/kg by 230) for the chemotherapeutic agent being used. The standard clinical dose for a particular patient can easily be calculated based on that patient's specific circumstances and would form part of the day to day activities of the skilled person.

The time between administration of the chemotherapeutic agent and the G rich oligonucleotide is preferably between 0 and 24 hours, with either the chemotherapeutic or the G rich oligonucleotide being administered first. It is well within the skilled person's capabilities to construct a schedule of times for administering the chemotherapeutic and G rich oligonucleotide based on the needs of the patient and availability of appropriate resources.

The combination therapy will be administered in a course of treatment. The exact frequency of treatment administration within the course and length of the course as a whole will depend upon the particular chemotherapeutic agent being used and the circumstances of the individual patient. It is entirely within the scope of a skilled person's abilities to be able to determine the appropriate length and frequency of treatment.

### Example 4

### Administration of Combination Therapy in Cancer Treatment Using an Intravenous Infusion

AS1411 is given to patients via intravenous infusion over a period of 7 days. The daily amount to be administered to the patient is calculated based on dose in mg/kg and the patient weight.

Fresh solutions are prepared on each infusion day, by diluting AS1411 drug product into 5% dextrose within an infusion bag. Appropriate infusion bags are known to those skilled in the art. A fresh infusion bag is preferably prepared at the start of each 24-hour period. After calculation of the required dose of AS1411, an equivalent volume of dextrose should be removed from the bag, and the required dose of AS1411 added directly to the bag for a total final volume of 500 mL.

Once prepared, infusion bags containing AS1411 can be stored at +2° C. to +5° C. until administration. Drug can be prepared up to 6 hours prior to dosing.

Reconstituted AS1411 in 5% dextrose is administered at room temperature as soon as possible following reconstitution. The appropriate dose of AS1411 is administered as a 500 ml intravenous infusion. Infusion of AS1411 is as close to 24 hours as possible, accounting for changing of infusion bags, or clotting of infusion lines.

Cytarabine is given to patients 1.5 g/m² twice daily via intravenous infusion over a period of 2 hours for each of 4 days. The daily amount to be administered to the patient is calculated based on dose in g/m². Preparation of cytarabine is performed following supplier's instructions.

### Example 5

### Administration of GRO in Cancer Treatment Using an Ambulatory Device

Administration of AS1411 is performed using an ambulatory device, which allows improved patient mobility. Such an administration route is useful for, for example, treatment of a patient with renal cancer.

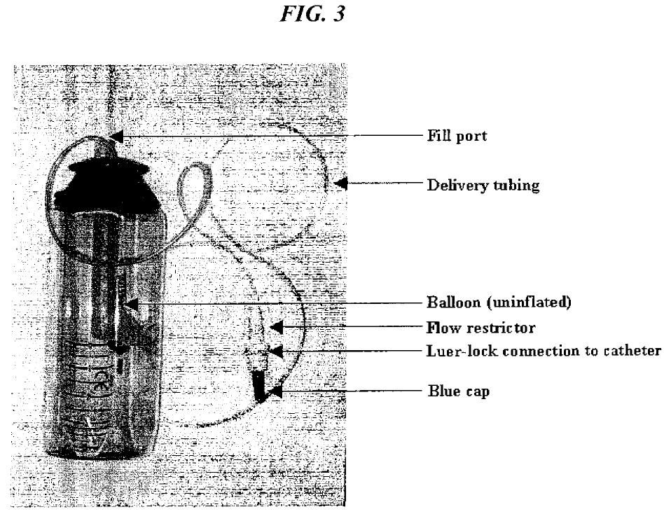

Ambulatory devices are well-known in the art of pharmacy and medicine and a skilled person would be able to select an appropriate device. A preferred device is the Baxter FOLFusor LV10 (Baxter Parkway, Deerfield, Ill. 60015-4625, USA; FIG. 3) which been used extensively in chemotherapy treatment, is non-allergenic, and supplies product at a rate of 10 ml/hour from a 240 ml reservoir. The FOLFusor is supplied in a “burn bag” to improve patient freedom and is replaced with a fresh, filled FOLFusor each day during the treatment cycle.

In the FOLFusor, product is introduced into a central elastomeric balloon via a syringe connected to a Fill Port located on the top of the device. The balloon is filled with 240 ml of AS1411. Having filled the device, the internal pressure within the balloon then drives the flow of product from the balloon through the delivery tubing via a luer-lock connector to the catheter. The flow rate is controlled by a restriction caused by a flow restrictor in the delivery tubing.

The flow rate accuracy is +/−10% and has been calibrated by Baxter using 5% dextrose. The FOLFusor must be filled to the nominal volume (240 ml) or the flow rate is reduced. A 5 micron in-line filter removes any particulates. There is no risk of air ingress as the FOLFusor is a closed system. If the FOLFusor dispenses all product and empties, there is some risk of blood tracking back up the tubing and causing a blockage. This can be removed with a heparin flush.

Details of the administration materials are:
AS1411 Drug Product concentrate, 20 mg/ml in 20 ml vials
Baxter FOLFusor LV10 (Baxter, catalogue no. 2C4063K)
Sterile syringe with Luer Lock Fitting, 100 ml capacity (e.g. Becton-Dickinson Plastipak)
Sterile Hypodermic needle
5% dextrose solution (Viaflex Container, Baxter, e.g. catalogue no. 2B0089)
Sterile Mixing vessel (preferably around 500 ml)

### (i) AS1411 Dose Calculation

AS1411 is delivered to the clinic as a concentrate in 20 ml vials at 20 mg/ml. AS1411 is first diluted into 5% dextrose at the clinic to give a final volume of 240 ml, the ratio of 5% dextrose to AS1411 is dependent on patient weight (see Table 1, below).

### (ii) AS1411 Solution Preparation

Using Table 1 as a guide, remove the required number of AS1411 vials from the refrigerator and allow to stand at room temperature for 1 hour. Using a sterile 100 ml syringe fitted with a hypodermic needle, withdraw the required volume of AS1411 concentrate from vials and add to the sterile mixing vessel. Using the same syringe, now withdraw the required volume of 5% dextrose from the Viaflex containers and add to the AS1411 concentrate in the mixing vessel. Swirl the container contents gently to mix. Note that Steps (ii) and (iii) must be carried out in a safety cabinet.

TABLE 1
Preparation Guidelines for AS1411 at varying patient weight
for 40 mg/kg dose

| Patient weight | Total g AS1411 per 24 | Volume | Volume 5% |
|---|---|---|---|
| (kg) | hours at 40 mg/kg | AS1411 (ml) | dextrose (ml) |
| 60 | 2.4 | 120 | 120 |
| 65 | 2.6 | 130 | 110 |
| 70 | 2.8 | 140 | 100 |
| 75 | 3.0 | 150 | 90 |
| 80 | 3.2 | 160 | 80 |
| 85 | 3.4 | 170 | 70 |
| 90 | 3.6 | 180 | 60 |
| 95 | 3.8 | 190 | 50 |
| 100 | 4.0 | 200 | 40 |
| 105 | 4.2 | 210 | 30 |
| 110 | 4.4 | 220 | 20 |
| 115 | 4.6 | 230 | 10 |
| 120 | 4.8 | 240 | 0 |

(iii) Addition of Drug to the FOLFusor.

The AS1411/dextrose solution is added to the FOLFusor using the 100 ml syringe screwed onto the Fill Port at the top of the device. Remove the hypodermic needle from the syringe and unscrew the cap from the Fill Port on the FOLFusor and retain in the cabinet. Remove the blue cap from the end of the delivery tube attached to the FOLFusor and retain in the cabinet (removal of the blue cap will allow air to be expelled from the device during priming). Fill the syringe with 100 ml of the AS1411 dextrose solution from the container and screw the syringe onto the Fill Port; slowly push the syringe plunder to transfer the solution into the device (the central balloon will inflate). Continue this process with additional syringe filling until 240 ml of the AS1411 dextrose solution is transferred to the FOLFusor (the balloon will now be fully inflated). Allow the drug solution to drip from the end of the delivery tube before replacing the blue cap.

### (iv) Connecting to the Catheter and Patient.

Now remove the filled FOLFusor from the safety cabinet. Using aseptic technique, remove the blue cap from the end of the delivery tube and attach to the catheter via the luer lock fitting. Allow drug solution to drip from the catheter before attaching to the patient.

### (v) Guidelines on Use.

The FOLFusor is then placed in a “burn bag” attached to the patient's waist. The FOLFusor should be kept at roughly the same height as the entry port into the patient. The flow rate decreases by 0.5% per 2.5 cm below this level, and increases by 0.5% per 2.5 cm above this level. Temperature and viscosity also impact the flow rate. A reduced temperature increases the viscosity and decreases the flow rate. A higher temp reduces the viscosity and increases the flow rate. 33.3° C. is the assumed temperature in the burn bag.

### Example 6

### Administration of Combination Therapy in a Xenograft Mouse Model

### Animals

The nu/nu MF1 mouse strain has been used previously at these laboratories and background data are available. A total of 50 female MF1 mice were obtained from the Biological Research Facility, SGHMS, London) in a weight range of 5 to 6 weeks of age (nominally 23-28 g). Mice were individually identified using a subcutaneously implanted microchip.

Animals were housed in solid bottom cages with filter tops that comply with the requirements of the Code of Practice for the housing and care of animals used in scientific procedures.

A commercially available rodent diet and sterile water were provided ad libitum throughout the study. Wood chips or shavings were provided as bedding for solid-bottom cages.

### Establishment and Measurement of Xenografts

The xenografts were prepared from the human AML cell line MV4-11 (DSMZ Number: CCL-102). Cells were grown in accordance with Standard Operating Procedures (SOP) and established methodology; suitable methods are known in the art. Cells were suspended at 2.0×10⁸ cells/ml in ice-cold PBS, mixed 1:1 with ice-cold Matrigel™ Matrix (BD Biosciences, Lot 61459) and kept on ice until the time of injection into animals. Mice were injected subcutaneously with 100 μl cell suspension on the right flank.

Animals were checked regularly until visible tumors appeared. Tumors were measured in accordance with the relevant SOP twice per week and tumor volume calculated using the formula: Volume=(π÷6000)×(Tumor Length×Tumor Width×Tumor Height).

Measurements commenced approximately one week before the day of treatment when the xenograft had reached a volume of >0.03 cm³.

Once the tumors were established, mice were allocated into groups using a randomisation procedure based on stratified xenograft volume. Measurements were recorded electronically using electronic calipers and the Trojan system for data capture.

Day 0 is defined as the day on which the average tumor volume is between 0.07 to 0.08 cm³. Relative tumor volume on Day 0 is defined as 1.

### Dosing

AS1411 (manufactured by Avecia, UK, batch number: AQV-07H-005; expiry date 02/2009) was given by continuous infusion using ALZET mini-osmotic pumps (Durect Corp. CA, USA). The mini-pumps were implanted subcutaneously on days 0 on the left flank of mice (opposite to the tumor location). Mice received a dose of 40 mg/kg/day with a dose-volume of 1 μl/h lasting for 7 days. The minipumps were then removed and a second drug-loaded minipump implantation in the same location to last a further 7 days.

The intraperitoneal route of administration of Cytarabine was selected. Mice were administered with Cytarabine (DBL, Australia; batch number: TO11966AA; expiry date 02/2009) at 80 mg/kg/daily for 5 consecutive days with a dose-volume of 8 μl/g body weight starting on day 0. After 2 days gap a second cycle of a similar 5-day dosing was initiated on day 7. Control mice were untreated.

### Study Design

Mice (n=10) were allocated to groups as follows:
Untreated controls
Cytarabine alone
AS1411 alone
Cytarabine+AS1411

### Determination of Synergism

The Fractional Product method was used to analyse the combination therapy (Yokohama et al., 2000, Cancer Res., 60:2190-6). According to this method, the effects of two drugs, when combined, can be calculated by multiplying the fractional tumor volume (treated volume/control volume on a given day) by each single drug. If the effect of the drugs acting simultaneously is equal to or larger than that calculated, it would be assumed that additivity, or synergism, respectively, has occurred.

### Results

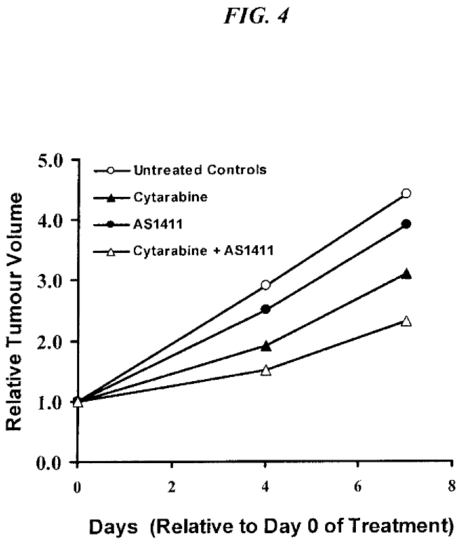

TABLE 2
Tumor volume change in xenograft mouse model at 7 days post-
treatment. The data in Table 2 is represented graphically in FIG. 4.
Volume of individual tumors at 7 days post-treatment in
xenograft mouse model (indicating tumor volume change)

| | | | Cytarabine |
|---|---|---|---|
| Control | Cytarabine | AS1411 | and AS1411 |
| 1.1 | 1.1 | 1.1 | 1.1 |
| 5.8 | 7.2 | 3.5 | 3.3 |
| 3.0 | 3.5 | 5.9 | 2.1 |
| 6.7 | 2.8 | 3.0 | 1.8 |
| 6.9 | 1.5 | 3.3 | 4.0 |
| 4.1 | 2.4 | 4.8 | 1.5 |
| 3.5 | 3.4 | 5.5 | 2.2 |
| 3.9 | 2.4 | 2.5 | 2.0 |
| 3.0 | 2.5 | 3.8 | 1.5 |
| 4.0 | 2.2 | 4.5 | 2.1 |
| 3.1 | | 2.5 | |
| Average = 4.4 | Average = 3.1 | Average = 3.9 | Average = 2.3 |

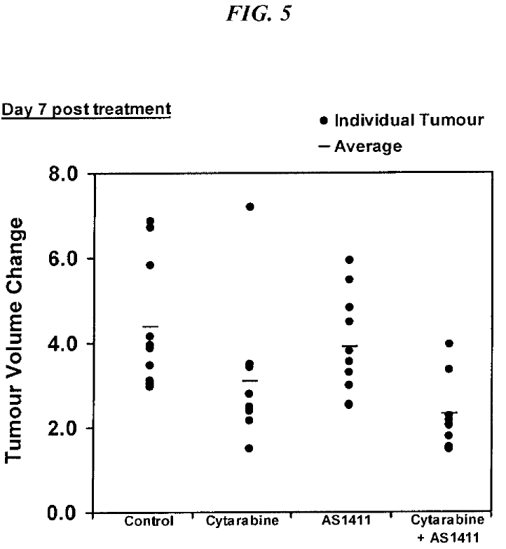

TABLE 3
Relative tumor volume at 4 and 7 days post-treatment (relative to
tumor volume at Day 0 of treatment) in xenograft mouse model.
The data in Table 3 is represented graphically in FIG. 5.

| | | | | Cytarabine |
|---|---|---|---|---|
| Days | Control | Cytarabine | AS1411 | and AS1411 |
| 0 | 1.000 | 1.000 | 1.000 | 1.000 |
| 4 | 2.900 | 1.900 | 2.500 | 1.500 |
| 7 | 4.400 | 3.100 | 3.900 | 2.300 |

TABLE 4
Analysis of the combination effect of Cytarabine (i.e. AraC) &
AS1411. Fractional Tumor Volume (FTV) relative to untreated
controls for AraC, AS1411 and combination treatments
(expected versus observed).

Combination Therapy

| Day | AraC | AS1411 | Expected | Observed | R |
|---|---|---|---|---|---|
| 4 | 0.713 | 0.848 | 0.605 | 0.618 | 0.979 |
| 7 | 0.844 | 0.915 | 0.772 | 0.650 | 1.187 |

### FTV (Fractional Tumor Volume) Calculation.

[Mean tumor volume test]/[Mean tumor volume control]

### Expected FTV Calculation:

[Mean FTV of Erbituxs]×[Mean FTV of AS1411]

(Note—Erbitux is a chimeric monoclonal antibody that blocks EGF HER1)

### Ratio (R) Calculation.

[Expected FTV]/[Observed FTV]

A combination therapy index R>1.0 indicates a synergistic effect.

A combination therapy index R<1.0 indicates a less than additive effect.

### Example 7

### Preferred Pharmaceutical Formulations and Modes and Doses of Administration

The polynucleotides and chemotherapeutics of the present invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The polynucleotides and chemotherapeutics of the present invention can be administered by a surgically implanted device that releases the drug directly to the required site. For example, Vitrasert releases ganciclovir directly into the eye to treat CMV retinitis. The direct application of this toxic agent to the site of disease achieves effective therapy without the drug's significant systemic side-effects.

Electroporation therapy (EPT) systems can also be employed for administration. A device which delivers a pulsed electric field to cells increases the permeability of the cell membranes to the drug, resulting in a significant enhancement of intracellular drug delivery.

Polynucleotides and chemotherapeutics of the invention can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin. The particles can be loaded or coated with drugs or genes or can simply act as “bullets” that generate pores in the skin through which the drugs can enter.

An alternative method of administration is the ReGel injectable system that is thermosensitive. Below body temperature, ReGel is an injectable liquid while at body temperature it immediately forms a gel reservoir that slowly erodes and dissolves into known, safe, biodegradable polymers. The active drug is delivered over time as the biopolymers dissolve.

Polynucleotides and chemotherapeutics of the invention can be introduced to cells by “Trojan peptides”. These are a class of polypeptides called penetratins which have translocating properties and are capable of carrying hydrophilic compounds across the plasma membrane. This system allows direct targeting of oligopeptides to the cytoplasm and nucleus, and may be non-cell type specific and highly efficient (Derossi et al., 1998, Trends Cell Biol., 8, 84-87).

Preferably, the pharmaceutical formulation of the present invention is a unit dosage containing a daily dose or unit, daily sub-dose or an appropriate fraction thereof, of the active ingredient.

The polypeptides, polynucleotides and antibodies of the invention can be administered by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the polypeptides, polynucleotides and antibodies of the invention can be administered alone but will generally be administered in admixture with a suitable pharmaceutical excipient diluent or carrier selected with regard to the intended route of administration and standard pharmaceutical practice.

As disclosed above, the polypeptides, polynucleotides and antibodies of the invention can also be administered parenterally, for example, intravenously, intra-arterially, intraperitoneally, intra-thecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Generally, in humans, continuous intravenous administration of the polynucleotides and chemotherapeutics of the invention is the preferred route.

For veterinary use, the polynucleotides and chemotherapeutics of the invention are administered as a suitably acceptable formulation in accordance with normal veterinary practice and the veterinary surgeon will determine the dosing regimen and route of administration which will be most appropriate for a particular animal.

The formulations of the pharmaceutical compositions of the invention may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question.

### Example 8

### Exemplary Pharmaceutical Formulations

Whilst it is possible for G-rich polynucleotides (or aptamer analogs) and chemotherapeutics of the invention to be administered alone, it is preferable to present it as a pharmaceutical formulation, together with one or more acceptable carriers. The carrier(s) must be “acceptable” in the sense of being compatible with the compound of the invention and not deleterious to the recipients thereof. Typically, the carriers will be water or saline which will be sterile and pyrogen-free.

The following examples illustrate pharmaceutical formulations according to the invention in which the active ingredient is a polynucleotide and/or chemotherapeutic of the invention.

### Example 8A

### Ophthalmic Solution

| | | |
|---|---|---|
| Active ingredient | 0.5 | g |
| Sodium chloride, analytical grade | 0.9 | g |
| Thiomersal | 0.001 | g |
| Purified water to | 100 | ml |

| | |
|---|---|
| pH adjusted to | 7.5 |

### Example 8B

### Capsule Formulations

### Formulation A

A capsule formulation is prepared by admixing the ingredients of Formulation D in Example C above and filling into a two-part hard gelatin capsule. Formulation B (infra) is prepared in a similar manner.

### Formulation B

| | mg/capsule |
|---|---|
| Active ingredient | 250 |
| Lactose B.P. | 143 |
| Sodium Starch Glycolate | 25 |
| Magnesium Stearate | 2 |
| | 420 |

### Formulation C

| | mg/capsule |
|---|---|
| Active ingredient | 250 |
| Macrogol 4000 BP | 350 |
| | 600 |

Capsules are prepared by melting the Macrogol 4000 BP, dispersing the active ingredient in the melt and filling the melt into a two-part hard gelatin capsule.

### Formulation D

| | mg/capsule |
|---|---|
| Active ingredient | 250 |
| Lecithin | 100 |
| Arachis Oil | 100 |
| | 450 |

Capsules are prepared by dispersing the active ingredient in the lecithin and arachis oil and filling the dispersion into soft, elastic gelatin capsules.

### Formulation E (Controlled Release Capsule)

The following controlled release capsule formulation is prepared by extruding ingredients a, b, and c using an extruder, followed by spheronisation of the extrudate and drying. The dried pellets are then coated with release-controlling membrane (d) and filled into a two-piece, hard gelatin capsule.

| | mg/capsule |
|---|---|
| Active ingredient | 250 |
| Microcrystalline Cellulose | 125 |
| Lactose BP | 125 |
| Ethyl Cellulose | 13 |
| | 513 |

### Example 8C

### Injectable Formulation

| | | |
|---|---|---|
| Active ingredient | 0.200 | g |
| Sterile, pyrogen free phosphate buffer (pH 7.0) to | 10 | ml |

The active ingredient(s) is dissolved in most of the phosphate buffer (35-40° C.), then made up to volume and filtered through a sterile micropore filter into a sterile 10 ml amber glass vial (type 1) and sealed with sterile closures and overseals.

Alternatively, the formulation may contain the following:
Potassium phosphate dibasic USP Quality (EMD Chemicals Inc, New Jersey 08027, USA) to pH 7.4;
Potassium phosphate monobasic USP Quality (EMD Chemicals Inc) to pH 7.4;
Water for Injection to 20 ml;
AS1411 400 mg

The weights of these materials used in each batch will depend on batch size. For example, the following could be used to give a batch size yielding approximately 1370 vials containing 20 ml at 20 mg/ml AS1411:
AS1411 528.5 g;
Potassium phosphate dibasic 39.8 g;
Potassium phosphate monobasic 8.2 g;
Water for Injection to 28339.8 g;
the formulation is mixed with 5% dextrose (Baxter) at the clinic.

### Example 8D

### Intramuscular Injection

| | | |
|---|---|---|
| Active ingredient | 0.20 | g |
| Benzyl Alcohol | 0.10 | g |
| Glucofurol 75 ® | 1.45 | g |
| Water for Injection q.s. to | 3.00 | ml |

The active ingredient(s) is dissolved in the glycofurol. The benzyl alcohol is then added and dissolved, and water added to 3 ml. The mixture is then filtered through a sterile micropore filter and sealed in sterile 3 ml glass vials (type 1).

### Example 8E

### Syrup Suspension

| | | |
|---|---|---|
| Active ingredient | 0.2500 | g |
| Sorbitol Solution | 1.5000 | g |
| Glycerol | 2.0000 | g |
| Dispersible Cellulose | 0.0750 | g |
| Sodium Benzoate | 0.0050 | g |
| Flavour, Peach 17.42.3169 | 0.0125 | ml |
| Purified Water q.s. to | 5.0000 | ml |

The sodium benzoate is dissolved in a portion of the purified water and the sorbitol solution added. The active ingredient is added and dispersed. In the glycerol is dispersed the thickener (dispersible cellulose). The two dispersions are mixed and made up to the required volume with the purified water. Further thickening is achieved as required by extra shearing of the suspension.

### Example 8F

### Suppository

| | mg/suppository |
|---|---|
| Active ingredient (63 μm)* | 250 |
| Hard Fat, BP (Witepsol H15 - Dynamit Nobel) | 1770 |
| | 2020 |

*The active ingredient(s) is used as a powder wherein at least 90% of the particles are of 63 μm diameter or less.

One fifth of the Witepsol H15 is melted in a steam-jacketed pan at 45° C. maximum. The active ingredient is sifted through a 200 μm sieve and added to the molten base with mixing, using a silverson fitted with a cutting head, until a smooth dispersion is achieved. Maintaining the mixture at 45° C., the remaining Witepsol H15 is added to the suspension and stirred to ensure a homogenous mix. The entire suspension is passed through a 250 μm stainless steel screen and, with continuous stirring, is allowed to cool to 40° C. At a temperature of 38° C. to 40° C. 2.02 g of the mixture is filled into suitable plastic moulds. The suppositories are allowed to cool to room temperature.

### Example 8G

### Pessaries

| | mg/pessary |
|---|---|
| Active ingredient | 250 |
| Anhydrate Dextrose | 380 |
| Potato Starch | 363 |
| Magnesium Stearate | 7 |
| | 1000 |

The above ingredients are mixed directly and pessaries prepared by direct compression of the resulting mixture:

### Example 8H

### Creams and Ointments

Described in Remington, The Science and Practise of Pharmacy, 19^{th} ed., The Philadelphia College of Pharmacy and Science, ISBN 0-912734-04-3.

### Example 8I

### Microsphere Formulations

The compounds of the invention may also be delivered using microsphere formulations, such as those described in Cleland (1997, Pharm. Biotechnol. 10:1-43; and 2001, J. Control. Release 72:13-24).

1. A method for treating a disease characterised by malignant, dysplastic, and/or hyperproliferative cells comprising exposing the malignant, dysplastic, and/or hyperproliferative cells to a combination of a G-rich oligonucleotide having the sequence of one of SEQ IDs Nos. 1 to 18 or an aptamer analog thereof and the chemotherapeutic agent cytarabine; wherein the G-rich oligonucleotide and the chemotherapeutic agent are administered in combination with one another.

2. The method as claimed in claim 1 wherein the administration of the G-rich oligonucleotide precedes treatment with the chemotherapeutic agent.

3. The method as claimed in claim 1 wherein the chemotherapeutic agent treatment precedes treatment with the G-rich oligonucleotide.

4. The method as claimed in claim 1 wherein the G-rich oligonucleotide and the chemotherapeutic agent are administered simultaneously.

5. The method as claimed in claim 1 wherein the G-rich oligonucleotide has the sequence of SEQ ID NO: 12 or the aptamer analog thereof.

6. The method as claimed in claim 1 wherein the G-rich oligonucleotide has a 3′ end and a 5′ end, and one or both of the 3′ and 5′ ends have been modified to alter a property of the G-rich oligonucleotide.

7. The method as claimed in claim 1 wherein the tumor is associated with at least one of the following disorders: acute myelogenous leukaemia, acute myeloid leukaemia (AML), acute lymphoblastic leukaemia (ALL), chronic myelogenous leukemia (CML), lymphomas, non-Hodgkin's lymphoma, and solid tumors including squamous cell carcinoma (such as head and neck cancer, and/or squamous cell carcinoma of the head and neck).

8. A pharmaceutical composition comprising a G rich oligonucleotide having the sequence of one of SEQ ID NO: 1 to 18 or an aptamer analog thereof and cytarabine in conjunction with a pharmaceutically acceptable excipient, diluent or carrier.

9. The pharmaceutical composition as claimed in claim 8 wherein the G-rich oligonucleotide has the sequence of SEQ ID NO: 12 or the aptamer analog thereof.

10. The method as claimed in claim 8 wherein the G-rich oligonucleotide has a 3′ end and a 5′ end, and one or both of the 3′, and 5′, ends have been modified to alter a property of the G-rich oligonucleotide.

11. A kit of parts comprising:
a G-rich oligonucleotide having the sequence selected from SEQ ID NO: 1 to 18 or an aptamer analog thereof;
cytarabine; and
instructions for their use.

12. The kit as claimed in claim 11 further comprising:
a system for administering the G-rich oligonucleotide and/or cytarabine to a patient.

13. The kit as claimed in claim 11 wherein the G-rich oligonucleotide and cytarabine are provided separately.

14. The kit as claimed in claim 11 wherein the G-rich oligonucleotide and cytarabine are provided as an admixture.

15. The kit as claimed in any of claims 11 wherein the G-rich oligonucleotide has the sequence of SEQ ID NO: 12 or the aptamer analog thereof.

16. The kit as claimed in claim 11 wherein the G-rich oligonucleotide has a 3′ end and a 5′ end, and one or both of the 3′ and 5′ ends have been modified to alter a property of the G-rich oligonucleotide.

17. A method for inhibiting the proliferation of malignant, dysplastic, and/or hyperproliferative cells in a subject, said method comprising administering to the subject a therapeutically effective amount of a G-rich oligonucleotide having the sequence of one of SEQ IDs Nos. 1 to 18 or an aptamer analog thereof in combination with the chemotherapeutic agent cytarabine.

18. The method as claimed in claim 17 wherein the administration of the G-rich oligonucleotide precedes treatment with the chemotherapeutic agent.

19. The method as claimed in claim 18 wherein the chemotherapeutic agent treatment precedes treatment with the G-rich oligonucleotide.

20. The method as claimed in claim 19 wherein both the G-rich oligonucleotide and the chemotherapeutic agent are administered simultaneously.

21. The method as claimed in claims 17 wherein the G-rich oligonucleotide has the sequence of SEQ ID NO: 12 or the aptamer analog thereof.

22. The method as claimed in claims 17 wherein the G-rich oligonucleotide has a 3′ end and a 5′ end, and one or both of the 3′ and 5′ ends have been modified to alter a property of the G-rich oligonucleotide.

23. The method as claimed in claims 17 wherein the malignant, dysplastic, and/or hyperproliferative cells are associated with at least one of the following disorders: acute myelogenous leukaemia, acute myeloid leukaemia (AML), acute lymphoblastic leukaemia (ALL), chronic myelogenous leukemia (CML), lymphomas, non-Hodgkin's lymphoma, and solid tumors including squamous cell carcinoma (such as head and neck cancer, and/or squamous cell carcinoma of the head and neck).


SEQUENCE LISTING (18 sequences)

SEQ ID NO: 1 - DNA, 14 bp, Artificial Sequence
  GRO14A
gttgtttggggtgg

SEQ ID NO: 2 - DNA, 15 bp, Artificial Sequence
  GRO15A
gttgtttggggtggt

SEQ ID NO: 3 - DNA, 25 bp, Artificial Sequence
  GRO25A
ggttggggtgggtggggtgggtggg

SEQ ID NO: 4 - DNA, 28 bp, Artificial Sequence
  GRO28A
tttggtggtggtggttgtggtggtggtg

SEQ ID NO: 5 - DNA, 29 bp, Artificial Sequence
  GRO29A
tttggtggtggtggttgtggtggtggtgg

SEQ ID NO: 6 - DNA, 29 bp, Artificial Sequence
  GRO29-2
tttggtggtggtggttttggtggtggtgg

SEQ ID NO: 7 - DNA, 29 bp, Artificial Sequence
  GRO29-3
tttggtggtggtggtggtggtggtggtgg

SEQ ID NO: 8 - DNA, 29 bp, Artificial Sequence
  GRO29-5
tttggtggtggtggtttgggtggtggtgg

SEQ ID NO: 9 - DNA, 13 bp, Artificial Sequence
  GRO29-13
tggtggtggtggt

SEQ ID NO: 10 - DNA, 11 bp, Artificial Sequence
  GRO11A
ggtggtggtgg

SEQ ID NO: 11 - DNA, 14 bp, Artificial Sequence
  GRO14C
ggtggttgtggtgg

SEQ ID NO: 12 - DNA, 26 bp, Artificial Sequence
  GRO26B
ggtggtggtggttgtggtggtggtgg

SEQ ID NO: 13 - DNA, 56 bp, Artificial Sequence
  GRO56A
ggtggtggtggttgtggtggtggtggttgtggtggtggtggttgtggtggtggtgg

SEQ ID NO: 14 - DNA, 32 bp, Artificial Sequence
  GRO32A
ggtggttgtggtggttgtggtggttgtggtgg

SEQ ID NO: 15 - DNA, 32 bp, Artificial Sequence
  GRO32B
tttggtggtggtggttgtggtggtggtggttt

SEQ ID NO: 16 - DNA, 29 bp, Artificial Sequence
  GRO29-6
ggtggtggtggttgtggtggtggtggttt

SEQ ID NO: 17 - DNA, 28 bp, Artificial Sequence
  GRO28B
tttggtggtggtggtgtggtggtggtgg

SEQ ID NO: 18 - DNA, 10 bp, Artificial Sequence
  GRO13A
tggtggtggt